(19) United States
(12) Patent Application Publication
(10) Pub. No.: US 2026/0354881 A1
(43) Pub. Date: Nov. 12, 2026

(54) ELECTROPORATION FOR SENSATION REDUCTION

(71) Applicant: Medtronic, Inc., Minneapolis, MN (US)

(72) Inventors: Matthew J. Hoffman, St. Paul, MN (US); Lars M. Mattison, Blaine, MN (US); Brian T. Howard, Minneapolis, MN (US); Kevin R. Seifert, Forest Lake, MN (US); Daniel W. Celotta, Circle Pines, MN (US)

(21) Appl. No.: 19/124,981

(22) PCT Filed: Oct. 26, 2023
(86) PCT No.: PCT/US2023/077975

§ 371 Date: Apr. 28, 2025

Related U.S. Application Data
(60) Provisional application No. 63/381,489, filed on Oct. 28, 2022.

(51) Int. Cl. A61B 18/00; A61B 17/00; A61B 90/00

(52) U.S. Cl. CPC A61B 18/00; A61B 90/04; A61B 2017/00557; A61B 2017/00889; A61B 2017/00893; A61B 2018/00178; A61B 2018/00434; A61B 2018/00577; A61B 2018/00613; A61B 2090/0436

(57) ABSTRACT

A medical device includes an elongated structure. The elongated structure is configured to be navigated from an access point of a patient to an implantation site within the patient. At least one electrode is carried on a distal portion of the elongated structure. The at least one electrode is configured, when proximate to the implantation site, to be oriented relative to a heart of the patient. The at least one electrode is configured to deliver electroporation energy to electroporate tissue proximate a ribcage of the patient and anterior to the heart.

This application claims the benefit of priority from U.S. Provisional Patent Application Ser. No. 63/381,489, filed Oct. 28, 2022, the entire content of which is incorporated herein by reference.

### TECHNICAL FIELD

This disclosure relates generally to implantable medical devices and, more particularly, to devices configured to deliver electroporation energy in the vicinity of location for an implantable medical lead.

### BACKGROUND

Medical device systems have been devised to provide electrical stimulation therapy without placing implantable medical leads within the heart or attaching implantable medical leads directly to the heart. These medical device systems may provide, for example, bradycardia pacing, anti-tachyarrhythmia pacing (ATP), post-shock pacing or other types of pacing to the heart from a non-transvenous or non-intracardiac location, such as from a location outside of the heart. In some patients, the medical device system implanted within the patient may also provide cardioversion or defibrillation therapy to the heart of the patient to terminate certain types of tachyarrhythmias, such as ventricular tachycardia (VT) or ventricular fibrillation (VF) to prevent sudden cardiac death (SCD).

### SUMMARY

Medical device systems, such implantable medical device systems or partially implantable medical device systems, configured to provide electrical stimulation therapy using electrodes outside of the heart may result in the patient experiencing sensation (e.g., paresthesia, pain, etc.) during the delivered stimulation. In the case of an implantable medical device (IMD) system configured to deliver pacing therapy to the heart of a patient, e.g., bradycardia pacing, anti-tachyarrhythmia pacing (ATP), post-shock pacing, pause prevention pacing, or other types of pacing, from an intrathoracic location, stimulation of skeletal muscles and intercostal nerves (and/or any other muscle tissue and nerve tissue) may occur proximate the electrodes of the lead or device delivering the therapy.

In accordance with techniques of this disclosure, an electroporation device may deliver electroporation energy to tissue responsible for sensation during pacing to reduce or eliminate sensation. For instance, delivering electroporation energy to the tissue may physiologically modify the cells of the tissue to which the energy is applied. The electroporation device may apply reversible electroporation energy or irreversible electroporation (IRE) energy. As a result of being subjected to the electrical pulses, sensation during pacing by the electroporated cells may be reduced or eliminated.

In some examples, a medical device comprises: an elongated structure configured to be navigated from an access point of a patient to an implantation site within the patient; and at least one electrode carried on a distal portion of the elongated structure, wherein the at least one electrode is configured, when proximate to the implantation site, to be oriented relative to a heart of the patient, and wherein the at least one electrode is configured to deliver electroporation energy to electroporate tissue proximate a ribcage of the patient and anterior to the heart.

In some examples, a medical system comprises: an introducer defining a lumen; an implantable medical lead configured to be navigated from an access point of a patient to an implantation site within the patient using the introducer, wherein the lumen is sized to allow insertion of the implantable medical lead into the introducer; and at least one electrode carried on a distal portion of the implantable medical lead, wherein the at least one electrode is configured, when proximate to the implantation site, to be oriented relative to a heart of the patient, and wherein the at least one electrode is configured to deliver electroporation energy to electroporate tissue proximate a ribcage of the patient and anterior to the heart.

In some examples, a method comprises: delivering, by at least one electrode carried on a distal portion of an elongated structure navigated from an access point of a patient to an implantation site within the patient, electroporation energy to electroporate tissue proximate a ribcage of the patient and anterior to the heart, wherein the at least one electrode is oriented relative to the heart of the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example implantable medical system in accordance with techniques of this disclosure.

FIG. 2 is a conceptual diagram illustrating an example lead in accordance with techniques of this disclosure.

FIG. 3 is a conceptual diagram illustrating an example lead carrying a shield in accordance with techniques of this disclosure.

FIGS. 4A-4B are conceptual diagrams illustrating an example lead carrying a balloon in accordance with techniques of this disclosure.

FIG. 5 is a conceptual diagram illustrating an example implantable medical system comprising an introducer in accordance with techniques of this disclosure.

FIG. 6 is a conceptual diagram illustrating an example implantable medical system comprising an external electrode in accordance with techniques of this disclosure.

FIG. 7 is a block diagram illustrating an example configuration of an electroporation device in accordance with techniques of this disclosure.

FIG. 8 is a flow diagram of an example technique for using an implantable medical system in accordance with techniques of this disclosure.

FIG. 9 is a flow diagram of an example technique for using an implantable medical system in accordance with techniques of this disclosure.

FIG. 10 is a flow diagram of an example technique for using an implantable medical system in accordance with techniques of this disclosure.

FIG. 11 is a flow diagram of an example technique for using an implantable medical system in accordance with techniques of this disclosure.

### DETAILED DESCRIPTION

As used herein, electroporation refers to a phenomenon that causes cell membranes to become “leaky” (that is, permeable for molecules for which the cell membrane may otherwise be impermeable or semipermeable). Electroporation, which may also be referred to as electropermeabilization, pulsed electric field treatment, non-thermal irreversible electroporation, irreversible electroporation, high frequency irreversible electroporation, nanosecond electroporation, or nanoelectroporation, may involve the application of high-amplitude pulses to cause physiological modification (i.e., permeabilization) of the cells of the tissue to which the energy is applied. These pulses may be short (e.g., nanosecond, microsecond, or millisecond pulse width, such as about 100 nanoseconds to about 20 milliseconds) in order to allow the application of high voltage (e.g., about 100 to 5000 volts), high current (e.g., 20 or more amps) without long duration(s) of electrical current flow that may otherwise cause significant tissue heating and muscle stimulation. In some examples, the number of pulses per second may be from about 1 to about 500. The pulsed electric energy may induce the formation of microscopic defects that result in hyperpermeabilization of the cell membrane. Depending on the characteristics of the electrical pulses, an electroporated cell can survive electroporation, referred to as “reversible electroporation,” or die, referred to as “irreversible electroporation” (IRE). Reversible electroporation may be used to transfer agents, including genetic material and other large or small molecules, into targeted cells for various purposes, including the alteration of the action potentials of cardiac myocytes.

In general, electrical stimulation of body tissue and organs may be used as a method of treating various conditions. Such stimulation is generally delivered by means of electrical contact between an implantable medical device (IMD) and a target site via one or more implantable electrodes, such as stimulation electrodes disposed on medical electrical leads, connected to the IMD. Leads typically include one or more stimulation electrodes disposed near a distal portion of the lead, which are positioned and/or anchored in proximity to the target site.

As discussed above, implantable cardioverter defibrillators (ICDs), including extravascular implantable cardioverter defibrillators (EV-ICDs), may deliver cardiac pacing in addition to anti-tachyarrhythmia shocks. In general, a patient may experience sensation (e.g., paresthesia, pain, etc.) during pacing delivered by an EV-ICD, such as via electrodes implanted substernally. Sensation may be due to stimulation of skeletal muscles and intercostal nerves (and/or any other muscle tissue and nerve tissue) proximate the electrodes of the lead. In accordance with techniques of this disclosure, an electroporation device may deliver electroporation energy to tissue responsible for sensation during pacing to reduce or eliminate sensation. For instance, delivering electroporation energy to the tissue may at least temporarily physiologically modify the cells of the tissue to which the energy is applied. In some examples, depending on the characteristics of the electrical pulses, the electroporated cells may be irreversibly electroporated such that sensation during pacing is reduced or eliminated entirely.

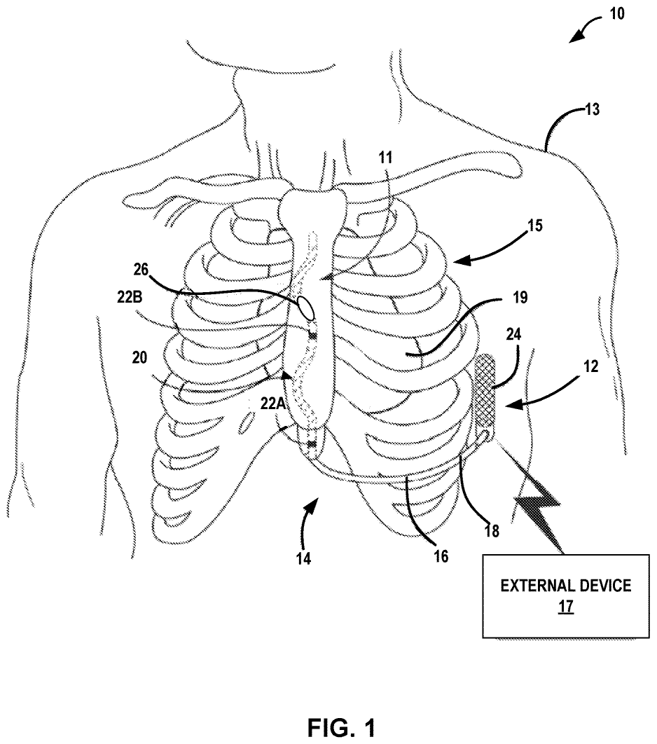

FIG. 1 is a conceptual diagram of an example medical system 10 (“system 10”) in accordance with techniques of this disclosure. System 10 is primarily described herein as an extravascular and/or extracardiac medical system, such as an EV-ICD system with a lead placed between sternum 11 and the pericardial surface. However, it should be understood that the techniques of this disclosure may apply to other medical device systems, such as intravascular and/or intracardiac medical systems, without limitation. For example, the techniques of this disclosure may apply to a pacemaker configured to deliver pacing therapy but not defibrillation therapy. Additionally, it should be understood that the techniques of this disclosure may apply to non-cardiac devices (e.g., neurostimulators, pelvic and gastric devices, etc.). In general, the techniques of this disclosure may apply to any medical device or system that delivers electrical therapy that may cause unintended sensation.

System 10 may include a medical device, such as an IMD 12 (e.g., an ICD). IMD 12 may include a signal generator configured to provide cardiac pacing and/or defibrillation therapy. As shown in FIG. 1, IMD 12 may be implanted subcutaneously on the left mid-axillary of a patient 13, superficially of the patient's ribcage 15. IMD 12 may be in wireless communication with an external device 17 (e.g., a computing device for use by a patient, a clinician, etc.) to transmit information to external device 17, be programmed by external device 17, etc. IMD 12 may be coupled to an elongated structure, such as an implantable medical lead 14 (“lead 14”). Lead 14 may be configured to be navigated from an access point of patient 13 to an implantation site within patient 13. Lead 14 may include a lead body 16 sized to be implanted extra-thoracically (outside the ribcage and sternum, e.g., subcutaneously or submuscularly) or intra-thoracically (e.g., beneath the ribcage or sternum, sometimes referred to as a “substernal” position) proximate a heart 19 of patient 13. For example, lead 14 may extend subcutaneously toward the center of the torso of patient 13, for example, toward the xiphoid process of patient 13.

At least a portion of a body 16 of lead 14 (“lead body 16”) may have a generally undulating shape or pattern (e.g., zig-zag, meandering, sinusoidal, serpentine, or other pattern). Additionally or alternatively, lead body 16 may have a generally uniform shape along the length of lead body 16. In another configuration, lead body 16 may have a flat, ribbon, or paddle shape along at least a portion of the length of the lead body 16. Other lead body 16 designs may be used without departing from the scope of this application. Lead body 16 of lead 14 may be formed from a non-conductive material, including silicone, polyurethane, fluoropolymers, mixtures thereof, and other appropriate materials, and shaped to form one or more lumens (not shown), however, the techniques are not limited to such constructions.

Lead body 16 may include a proximal portion 18 and a distal portion 20. Distal portion 20 may carry one or more electrodes configured to deliver electrical energy to the heart or sense electrical energy within the heart. Distal portion 20 may be anchored to a desired position within the patient, for example, substernally or subcutaneously by, for example, suturing distal portion 20 to the patient's musculature, tissue, or bone at the xiphoid process entry site. Alternatively, distal portion 20 may be anchored to the patient or through the use of a fixation mechanism, such as rigid tines, prongs, barbs, clips, screws, flanges, etc. For example, distal portion 20 may be anchored proximate a target site within patient 13.

In some examples, distal portion 20 of lead body 16 may be implanted within the anterior mediastinum. The anterior mediastinum may be viewed as being bounded laterally by the pleurae, posteriorly by the pericardium, and anteriorly by sternum 11. In some instances, the anterior wall of the anterior mediastinum may also be formed by the transversus thoracis and one or more costal cartilages. The anterior mediastinum includes a quantity of loose connective tissue (such as areolar tissue), some lymph vessels, lymph glands, substernal musculature (e.g., transverse thoracic muscle), branches of the internal thoracic artery, and the internal thoracic vein. In one example, distal portion 20 of lead body 16 may be implanted substantially within the loose connective tissue and/or substernal musculature of the anterior mediastinum.

In other examples, distal portion 20 of lead body 16 may be implanted in other extra-thoracic, intra-thoracic, including extravascular, extracardiac, or extra-pericardial locations, including the gap, tissue, or other anatomical features around the perimeter of and adjacent to the pericardium or other portion of the heart and not above sternum 11 or ribcage 15. As such, lead 14 may be implanted anywhere within the substernal space defined by the undersurface between sternum 11 and/or ribcage 15 and the body cavity.

Distal portion 20 may include or otherwise support (e.g., carry) one or more electrodes, such as electrodes 22A-22B (collectively, “electrodes 22”). Examples of electrodes 22 may include segmented electrodes, circumferential electrodes, ring electrodes, ribbon electrodes, short coil electrodes, paddle electrodes, hemispherical electrodes, directional electrodes, defibrillation electrodes, etc., and may be positioned at any position along distal portion 20.

Proximal portion 18 of lead body 16 may include one or more connectors to electrically couple lead 14 to IMD 12. In some examples, each of the electrodes 22 on distal portion 20 is electrically connected to a corresponding connector on proximal portion 18.

IMD 12 may include a housing 24 that forms a hermetic seal that protects components of IMD 12. Housing 24 of IMD 12 may be formed of a conductive material, such as titanium or titanium alloy, which may function as a housing electrode for a particular therapy vector between housing 24 and distal portion 20. The IMD 12 may also include a connector assembly that includes electrical feedthroughs through which electrical connections are made between the one or more connectors of lead 14 and electronic components included within housing 24. Housing 24 may contain circuitry, such as processing circuitry, memory circuitry, telemetry circuitry, sensing circuitry, therapy circuitry (which may include, for example, a pulse generator(s), transformer(s), capacitor(s), or the like), switching circuitry, power circuitry (capacitors and batteries), etc.

IMD 12 may generate and deliver electrical stimulation therapy, including traditional low voltage stimulation therapies (e.g., anti-tachycardia pacing, post-shock pacing, bradycardia pacing, or pacing used in conjunction with VF induction) as well as (optionally) traditional high voltage stimulation therapies (e.g., cardioversion or defibrillation shocks) via various electrode combinations or vectors.

IMD 12 may detect a ventricular tachyarrhythmia (e.g., VT or VF) based on signals sensed using electrodes 22. In response to detecting the tachyarrhythmia, IMD 12 may generate low voltage and/or high voltage electrical stimulation therapy and deliver the electrical stimulation therapy via electrodes 22. Additionally or alternatively, IMD 12 may deliver pacing (e.g., ATP or post-shock pacing). If high voltage therapy is necessary, IMD 12 may deliver a cardioversion/defibrillation shock (or multiple shocks) using electrodes 22 (and in some cases housing 24). IMD 12 may generate and deliver the pacing pulses to provide anti-tachycardia pacing (ATP), bradycardia pacing, post shock pacing, or other pacing therapies or combination of pacing therapies. In this manner, ATP therapy or post shock pacing (or other pacing therapy) may be provided in system 10 without entering the vasculature or the pericardial space, nor making intimate contact with the heart.

In some examples, lead 14 may include a balloon 26 located at distal portion 20. Balloon 26 may define an interior volume configured to receive an inflating medium (e.g., air, saline, or another medium), in turn resulting in inflation of balloon 26. Lead 14 may define an inflation lumen fluidly coupled to the interior volume and configured such that a clinician may deliver the inflating medium to the interior volume defined by balloon 26. The inflation lumen may extend from proximal portion 18 to distal portion 20 of lead body 16. An exterior surface of proximal portion 18 may define an opening to the inflation lumen.

As described above, a patient may experience sensation during pacing because of, for example, stimulation of skeletal muscles and intercostal nerves (and/or any other muscle tissue and nerve tissue) proximate electrodes 22 of lead 14. In accordance with techniques of this disclosure, system 10 may include an electroporation device (not shown) configured to deliver electroporation energy (e.g., via lead 14) to target tissue to at least temporarily modify the physiological characteristics of the cells of the tissue, thereby preventing undesirable sensation, while leaving other tissue (e.g., epicardium tissue) undamaged (e.g., not impacted, reversibly electroporated, etc.). In this way, the techniques of this disclosure may safely reduce discomfort experienced by the patient during treatment, improving patient outcomes.

The electroporation device may be electrically connected to lead 14. Lead 14 may be navigated to an implantation site within patient 13 such that electrodes 22 are proximate to the implantation site. When proximate to the implantation site, electrodes 22 may be oriented relative to heart 19 of patient 13. For instance, in examples where electrodes 22 are segmented electrodes (e.g., directional electrodes), electrodes 22 may be oriented toward a posterior sternal surface such that the electrical fields produced by electrodes 22 are simultaneously directed toward target tissue for ablation and away from heart 19.

The electroporation device may include a signal generator configured to provide electrical pulses to electrodes 22 to perform an electroporation procedure. For instance, the signal generator may be configured and programmed to deliver pulsed, high-voltage electric fields appropriate for achieving desired pulsed, high-voltage ablation via reversible electroporation, IRE, pulsed radiofrequency (RF) ablation, etc.

In the example of reversible electroporation, the pulsed-field energy may temporarily prevent the electroporated cells from experiencing sensation. In this way, a clinician may confirm the target tissue before causing the death of the tissue. Following confirmation of the target tissue, the clinician may induce the death of the reversibly electroporated cells. For example, system 10, such as lead 14, may deliver IRE or a therapy agent to cause cell death in reversibly electroporated cells, such as tissue proximate a ribcage of the patient. In some examples, the therapy agent may be preferentially placed on lead 14 to target the side of lead 14 closer to ribcage 15 of patient 13. Additionally or alternatively, a local or systemic injection of a therapy agent may cause cell death following the reversible electroporation energy, allowing for the same ablative effect. In some examples, the therapy agent may include an anti-inflammatory agent, an analgesic agent, a neurotoxin, an antimicrobial agent, etc. For example, the therapy agent may be Bleomyocin.

In the example of IRE, the pulsed-field energy may be sufficient to induce cell death for purposes of destroying the ability of the so-ablated tissue to propagate or conduct electrical signals associated with undesirable sensation. The target tissue may include tissue proximate ribcage 15 of patient 13 and anterior to heart 19, such as intercostal nerves, transverse thoracic muscle tissue, etc.

Although the elongated structure is primarily described with respect to FIG. 1 as being an implantable medical lead, other examples of the elongated structure are contemplated by this disclosure. For instance, the elongated structure may be a catheter or an introducer configured to deliver electroporation energy to electroporate tissue responsible for sensation during pacing. Additionally, while the electroporation device is primarily described herein as being outside the body of patient 13, IMD 12 may additionally or alternatively include a signal generator configured to provide electrical pulses for performing an electroporation procedure in accordance with techniques of this disclosure.

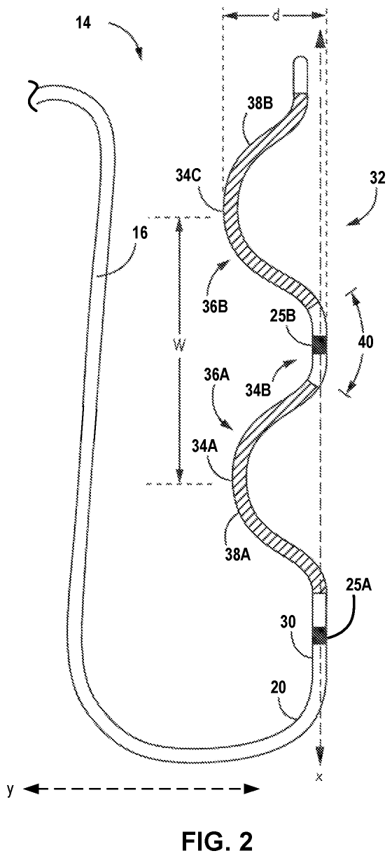

FIG. 2 is conceptual diagram of lead 14. As shown in FIG. 2, distal portion 20 may define an undulating configuration 32 distal to a substantially linear portion 30 (“linear portion 30”). In particular, distal portion 20 may define an undulating pattern, e.g., (zig-zag, meandering, sinusoidal, serpentine, or other pattern) as it extends toward the distal end of distal portion 20. Undulating configuration 32 may be substantially disposed in a plane defined by the longitudinal axis (“x”) and a transverse axis (“y”). In some examples, lead body 16 may not have linear portion 30 as it extends distally, but instead undulating configuration 32 may begin immediately after the bend.

Undulating configuration 32 may include a plurality of peaks along the length of distal portion 20, such as peaks 34A-34C (collectively, “peaks 34”). Undulating configuration 32 may include any number of peaks 34. For example, the number of peaks 34 may be fewer or greater than three depending on the frequency of the undulation configuration 32. Undulating configuration 32 may define a peak-to-peak amplitude or distance “d,” (shown in FIG. 2), which may be variable or constant along the length of undulating configuration 32. As shown in FIG. 2, undulating configuration 32 may define a substantially sinusoidal configuration, with a constant peak-to-peak distance “d” of approximately 2.0-5.0 centimeters (cm). Undulating configuration 32 may also define a peak-to-peak width “w,” (shown in FIG. 2), which may also be variable or constant along the length of undulating configuration 32. In other instances, undulating configuration 32 may define other shapes and/or patterns, e.g., S-shapes, wave shapes, or the like.

Distal portion 20 may include defibrillation electrodes, such as defibrillation electrodes 36A-36B (collectively, “defibrillation electrodes 36”). Defibrillation electrodes may be examples of electrodes 22 shown in FIG. 1. Defibrillation electrodes 36 may be configured to deliver a cardioversion/defibrillation shock. Defibrillation electrodes 36 may include a plurality of sections or segments, such as segments 38A-38B (collectively, “segments 38”), spaced a distance apart from each other along the length of distal portion 20. Segments 38 may be a disposed around or within distal portion 20 of lead body 16, or alternatively, may be embedded within the wall of lead body 16. In one configuration, segments 38 may be a coil electrode formed by a conductor. The conductor may be formed of one or more conductive polymers, ceramics, metal-polymer composites, semiconductors, metals or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of segments 38 may be a flat ribbon electrode, a paddle electrode, a braided or woven electrode, a mesh electrode, a directional electrode, a patch electrode or another type of electrode configured to deliver a cardioversion/defibrillation shock to the patient's heart.

Segments 38 may be electrically connected to one or more conductors, which may be disposed in the body wall of lead body 16 or may alternatively be disposed in one or more insulated lumens (not shown) defined by lead body 16. In an exemplary configuration, each of segments 38 is connected to a common conductor such that a voltage may be applied simultaneously to all segments 38 to deliver a defibrillation shock to a patient's heart. In other configurations, segments 38 may be attached to separate conductors such that each of segments 38 may apply a voltage independent of other segments 38. For example, IMD 12 or lead 14 may include one or more switches or other mechanisms to electrically connect segments 38 together to function as a common polarity electrode such that a voltage may be applied simultaneously to all segments 38 in addition to being able to independently apply a voltage.

Distal portion 20 may define one or more gaps 40 between adjacent segments 38. Gaps 40 may define any length. In instances in which more than two segments 38 exist, each of gaps 40 may define the same or substantially the same length as other gaps 40 or may define a different length than other gaps 40 in distal portion 20. One or more electrodes be disposed within respective gaps 40. For example, electrodes 25A-25B (collectively, “electrodes 25”) may be disposed within respective gaps 40. Additionally or alternatively, electrodes 25 may be disposed along distal portion 20 of lead body 16 (e.g., proximal to segment 38A and/or distal to segment 38B). Electrodes 25 may be examples of electrodes 22 shown in FIG. 1.

Electrodes 25 and/or defibrillation electrodes 36 (e.g., via segments 38) may be configured to deliver electroporation energy in accordance with techniques of this disclosure.

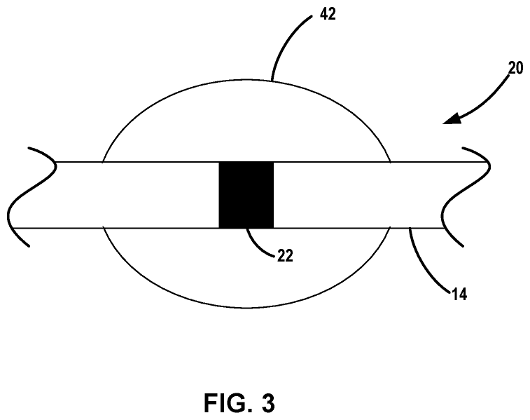

FIG. 3 is a conceptual diagram of a portion of lead 14 carrying a shield 42 in accordance with techniques of this disclosure. In some examples, distal portion 20 of lead body 16 may include one or more shields 42. Shield 42 may be configured to impede an electric field from delivery of an electrical therapy. For example, shield 42 may be positioned relative to electrodes 22 (e.g., electrodes 25, defibrillation electrodes 36, etc.). For example, shield 42 may cover or be otherwise disposed over a portion of an outer surface of electrodes 22. Shield 42 may not cover an entirety of the outer surface of electrodes 22. Pulsed electric energy delivered by lead 14 via electrodes 22 may result in an electrical field proximate electrodes 22 that “spreads” from electrodes 22. Shield 42 may impede the electrical field in directions from electrodes 22 toward shield 42, and allow the spread in directions from the electrode 22 away from shield 42. In this way, shield 42 may be configured to make electrodes 22 directional.

Shield 42 may be positioned between electrodes 22 and heart 6 such that shield 42 impedes delivery of electroporation energy from electrodes 22 toward heart 6. As a result, shield 42 may reduce the likelihood that the electrical field from electrodes 22 will electroporate cardiac tissue by redirecting the electrical field from heart 6 toward extracardiac tissue, such as skeletal tissue and intercostal nerves. Thus, in examples where electrodes 22 are circumferential electrodes or ring electrodes, shield 42 may be used to prevent unintended electroporation of heart 6 during an electroporation procedure in accordance with techniques of this disclosure.

As shown in FIG. 3, shield 42 may extend laterally away from electrodes 22, e.g., in a substantially planar manner, such that the dimensions of shield 42 in a plane are greater than those of electrodes 22 in the plane. In this manner, shield 42 may further (or more effectively) limit the directions, e.g., radial angles, of the spread of the electrical field generated by the pacing pulse (e.g., pulsed electric energy) from electrodes 22. The plane in which shield 42 extends laterally from electrodes 22 may be the same plane in which peaks 34 of the undulating configuration extend, or a substantially parallel plane. In some examples, such as that shown in FIG. 3, shield 42 may extend symmetrically from electrodes 22, e.g., be symmetrical about a longitudinal axis and/or a transverse axis of electrodes 22, such that electrodes 22 is substantially centered within the outer profile of shield 42 in the plane.

Shield 42 may be electrically insulative. In some examples, shield 42 may comprise a polymer, such as polyurethane. In some examples, shield 42 may be configured to be folded or wrapped around electrodes 22 for delivery via a lumen of an implant tool, and configured to elastically unfold or unwrap to a relaxed condition, e.g., such as the condition shown in FIG. 3, when released from the lumen. In some examples, shield 42 may comprise elastic or super-elastic polymer or metallic structures, e.g., Nitinol structures, to encourage the deployment of shield 42, support articulation of shield 42, and/or support shield 42 in the deployed, relaxed configuration. The deployed and/or articulated configuration may be substantially planar, as illustrated in FIG. 3, or may be non-planar. For example, portions of shield 42 spaced further away laterally from electrodes 22 may be situated more posteriorly than portions closer to electrodes 22, e.g., in the shape of a cup or bowl.

Such support structures may be partially or fully embedded within a primary material of shield 42, or attached to one or more outer surfaces of shield 42. In some examples, a support structure is located circumferentially around a perimeter of shield 42, e.g., spaced a greatest distance laterally from shield 42. However, other support structure locations are possible. For example, one or more support structures may extend in radial or lateral direction from electrodes 22.

Following electroporation in accordance with techniques of this disclosure, shield 42 may be repositioned on lead 14 and/or reconfigured to allow stimulation energy to reach the heart unimpeded by shield 42. For example, lead 14 may be withdrawn from the patient's body and shield 42 may be repositioned on lead 14 such that when electrodes 22 are navigated to an implantation site and oriented relative to heart 19, electrodes 22 are disposed between shield 42 and heart 19. In some examples, shield 42 may be removed or placed in a closed configuration (e.g., folded, wrapped, etc.). In some examples, instead of repositioning shield 42, lead 14 may be reoriented relative to heart 19, e.g., rotated about its longitudinal axis.

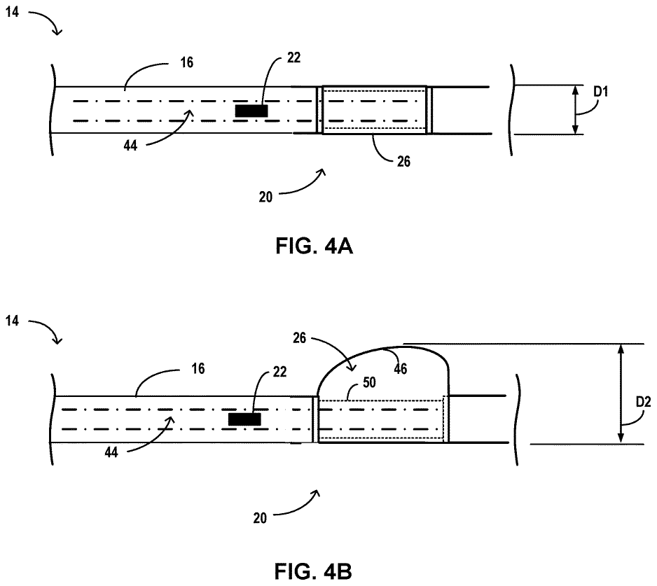

FIGS. 4A-4B are conceptual diagrams of lead 14 carrying balloon 26. Lead 14 may include balloon 26 located at distal portion 20. Balloon 26 may be affixed to distal portion 20. As shown in FIG. 4A, an inflation lumen 44 may extend to balloon 26, such that balloon 26 and inflation lumen 44 are in fluid communication.

In some examples, lead body 16 may be positioned within an introducer. In some examples, the introducer is a delivery catheter. The introducer may include an inner wall defining an introducer lumen and further includes an introducer opening to the introducer lumen. Lead 14 may be configured to translate through the introducer lumen to pass through the introducer opening when balloon 26 is in the deflated configuration.

In any case, balloon 26 may be expanded enroute to positioning distal portion 20 in the vicinity of a target site within patient 13. FIG. 4A illustrates balloon 26 in the deflated configuration and defining a maximum initial dimension D1 (e.g., an inner diameter). Lead 14 may define maximum initial dimension D1, for example, to allow lead body 16 to translate through an introducer lumen and introducer opening.

FIG. 4B illustrates lead 14 with balloon 26 in the inflated configuration. Balloon 26 may define an interior volume 46 configured to contain an inflating medium (e.g., air, saline, or another inflating medium) to cause balloon 26 to transition from the deflated configuration of FIG. 4A to the inflated configuration depicted in FIG. 4B. In some examples, interior volume 46 may be bound at least in part by an inner surface 46 of balloon 26 and an exterior surface 50 of distal portion 20. Lead body 16 may define an inflation lumen 44 configured to provide the inflating medium to interior volume 46. For instance, inflation lumen 44 may extend to interior volume 46, such that balloon 26 and interior volume 46 are in fluid communication.

In the inflated configuration, balloon 26 may define a maximum expanded dimension D2 (e.g., an inner diameter). The maximum expanded dimension D2 of the inflated configuration is greater than the maximum initial dimension D1 of the deflated configuration. When in the inflated configuration, balloon 26 may decrease the distance between electrodes 22 and ribcage 15 and increase the distance between electrodes 22 and heart 19. Consequently, when in an inflated configuration, balloon 26 may position electrodes 22 toward ribcage 15 and away from heart 19, advantageously reducing the likelihood of undesirably electroporating heart 19 when electroporating tissue responsible for undesirable sensation in accordance with techniques of this disclosure. In some examples, balloon 26 may be coextensive longitudinally with electrodes 22 and also act as a shield when in the inflated configuration.

It should be understood that any of the examples described herein may be combined without limitation and are contemplated by this disclosure. For example, lead 14 may simultaneously carry balloon 26 and shield 42 to prevent or reduce electrical stimulation (e.g., IRE) of unintended tissue.

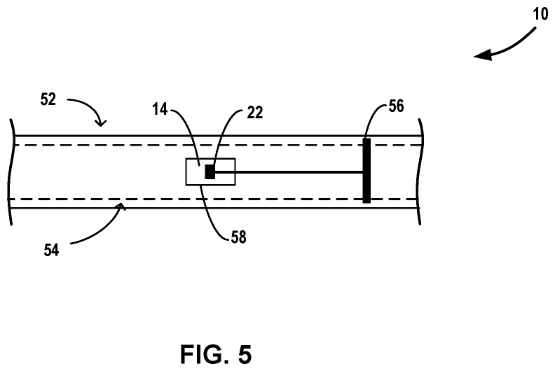

FIG. 5 is a conceptual diagram illustrating system 10 further comprising an introducer 52. Introducer 52 may define a lumen 54 sized to allow insertion of lead 14 into introducer 52. Introducer 52 may be configured to facilitate navigation of lead 14 from an access point of patient 13 to an implantation site within patient 13. In addition, introducer 52 may be configured to facilitate delivery of electroporation energy to electroporate tissue proximate ribcage 15 and anterior to heart 19. In some examples, introducer may support or otherwise include (e.g., carry) one or more introducer electrodes 56 configured to deliver electroporation energy to electroporate the tissue proximate ribcage 15 and anterior to heart 19. Introducer electrodes 56 may be configured to be oriented toward a posterior sternal surface. In some examples, introducer electrodes 56 may be electrically connected to a signal generator, and introducer electrodes 56 may deliver electroporation energy to the target tissue. In other examples, electrodes 22 (e.g., an electrode on lead 14) may be electrically connected to one or more of introducer electrodes 56 via a conductor configured to conduct the electroporation energy from electrodes 22 to introducer electrodes 56.

In some examples, introducer 52 may define at least one slot 58 that exposes electrodes 22 when electrodes 22 are proximate to the implantation site and oriented relative to heart 19. Additionally or alternatively, introducer 52 may define a porous structure that facilitates delivery of electroporation energy from electrodes 22.

In some examples, introducer 52 may be coupled at a proximal end to an electroporation device and be configured to deliver the electroporation energy introducer 52 receives from the electroporation device. Introducer 52 may be configured to deliver the electroporation energy in addition to or instead of lead 14. It should be understood that introducer 52 is one example of an elongated structure in accordance with techniques of this disclosure, and other tools configured to facilitate advancement of a lead or catheter toward a target site and to deliver electroporation energy are contemplated by this disclosure.

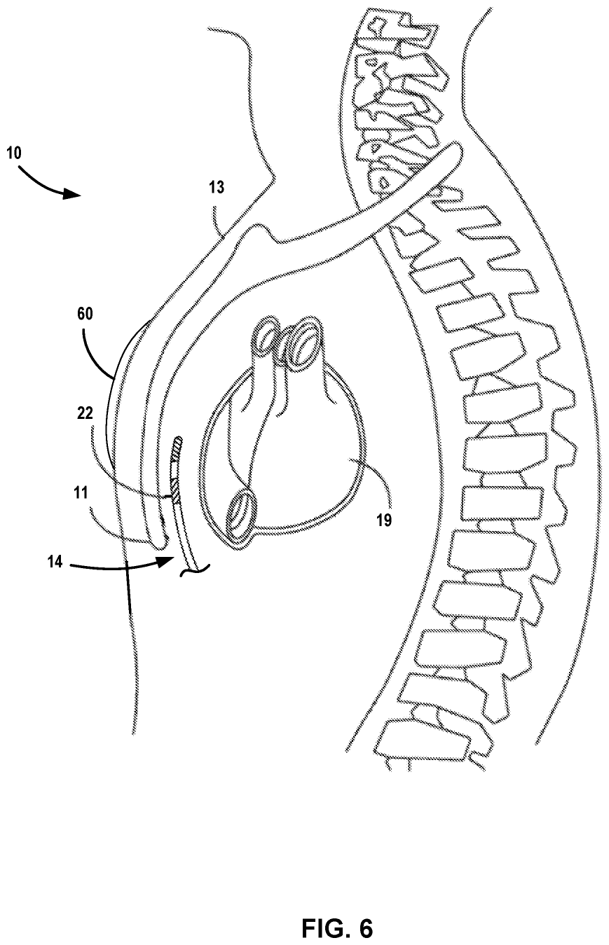

FIG. 6 is a conceptual diagram of system 10 further comprising an external electrode 60. External electrode 60 may be wearable by, e.g., attached to, patient 13. System 10 may perform IRE to tissue responsible for undesirable sensation in accordance with techniques of this disclosure using electrodes 22 of lead 14 and external electrode 60. In some examples, external electrode 60 may a removable pad or patch configured to be placed on the patient's body. External electrode 60 may be placed and replaced to electroporate various target tissues (e.g., various sites proximate to the sternum 11) for ablation.

It should be understood that any of the examples described herein may be combined without limitation and are contemplated by this disclosure. For example, system 10 may include external electrode 60, balloon 26, and shield 42 to perform IRE in accordance with techniques of this disclosure while preventing or reducing electrical stimulation of unintended tissue.

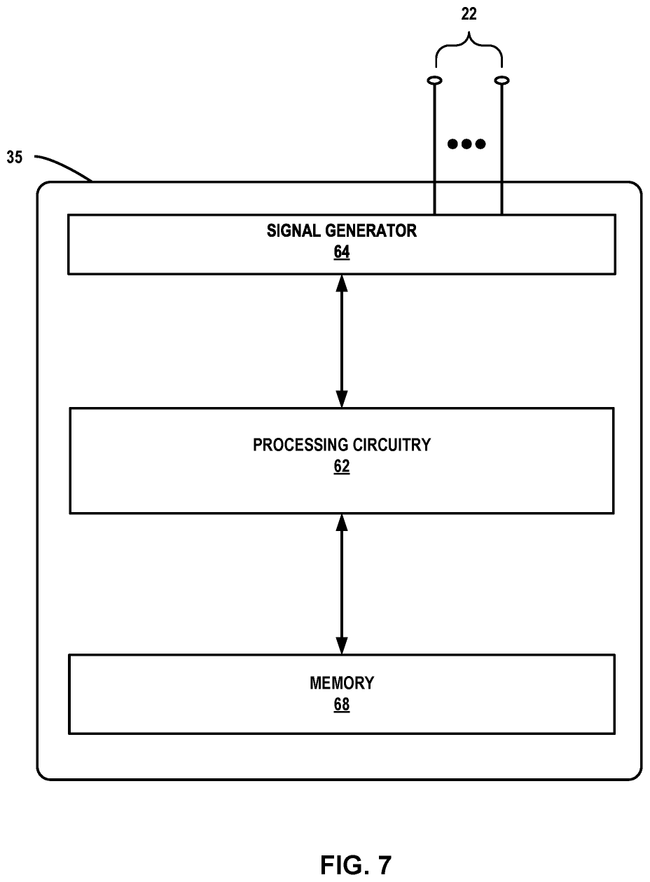

FIG. 7 is a block diagram illustrating an example configuration of an electroporation device 35 configured to configure electroporation energy in accordance with techniques of this disclosure. As shown in FIG. 7, electroporation device 35 includes processing circuitry 62, signal generator 64, and memory 68. Electroporation device 35 may be electrically connected to electrodes 22 via lead 14. For example, proximal portion 18 of lead body 16 may be electrically connected to electroporation device 35. In some examples, memory 68 includes computer-readable instructions that, when executed by processing circuitry 62, cause electroporation device 35 and processing circuitry 62 to perform various functions attributed herein to electroporation device 35 and processing circuitry 62. Memory 68 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 62 may include fixed function circuitry and/or programmable processing circuitry. Processing circuitry 62 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 62 may include multiple components, such as any combination of one or more microprocessors, one or more controllers, one or more DSPs, one or more ASICs, or one or more FPGAs, as well as other discrete or integrated logic circuitry. The functions attributed to processing circuitry 62 herein may be embodied as software, firmware, hardware or any combination thereof.

Signal generator 64 may be selectively coupled to electrodes 22. Signal generator 64 may be configured to provide electrical pulses to electrodes 22 to perform an electroporation procedure. For instance, signal generator 64 may be configured and programmed to deliver pulsed, high-voltage electric fields appropriate for achieving desired pulsed, high-voltage ablation via IRE and/or pulsed RF ablation. In some examples, signal generator 64 may be coupled to external electrode 60 (e.g., in addition to lead 14).

In general, IRE may be an acute procedure, meaning it may only need to be performed once to achieve the advantages disclosed herein. IRE may be performed at any time (e.g., perioperatively, postoperatively, etc.). However, IRE is primarily described herein as being performed perioperatively.

Signal generator 64 may provide electrical pulses to perform an electroporation procedure to extracardiac tissue within the cardiothoracic space, or other tissues within the body, such as renal tissue, or airway tissue. Electroporation utilizes high amplitude pulses to effectuate a physiological modification (i.e., permeabilization) of the cells to which the energy is applied. Such pulses may preferably be short (e.g., nanosecond, microsecond, or millisecond pulse width) in order to allow application of high voltage, high current (for example, 20 or more amps) without long duration of electrical current flow that results in significant tissue heating. In particular, the pulsed energy induces the formation of microscopic pores or openings in the cell membrane.

Signal generator 64 may be configured and programmed to deliver pulsed, high voltage electric fields appropriate for achieving desired pulsed, high voltage ablation (or pulsed field ablation). As a point of reference, the pulsed, high voltage, non-radiofrequency, ablation effects of the present disclosure may be distinguishable from DC current ablation, as well as thermally-induced ablation attendant with conventional RF techniques. For example, the pulse trains delivered by signal generator 64 may be delivered at a frequency less than 3 kHz, and in an exemplary configuration, 1 kHz, which is a lower frequency than radiofrequency treatments. The pulsed-field energy in accordance with the present disclosure may be sufficient to induce cell death for purposes of preventing sensory response to cardiac pacing or other electrical stimulation as described herein.

In some examples, electrodes 22 may deliver therapeutic biphasic pulses having a preprogrammed pattern and duty cycle. For example, each pulse cycle may include an applied voltage amplitude A, a pulse width B (in microseconds (μs)), an inter-phase delay C (in μs), an inter-pulse delay D (in μs), and a pulse cycle length E. In an exemplary configuration, the pulse width B may be 1-15 μs, the inter-phase delay C may be 0-4 μs, the inter-pulse delay D may be 5-30,000 μs, the pulse train may include 20-1000 pulses, and the applied voltage may be approximately 300-4000 V. In some examples, the pulse width may be set to 5 μs, the inter-phase delay may be 5 μs, the inter-pulse delay may be 800 μs, and the pulse train may include 80 pulses with an applied voltage of 700V. Such a pulse train when delivered from a bipolar electrode array may produce lesions in tissue in the range of approximately 2-3mm deep. Increased voltage may correspondingly increase the lesion depth. In another example, four pulse trains may be delivered at each target tissue site.

The pulsed field of energy may be delivered in a bipolar fashion, in monophasic or biphasic pulses. The application of biphasic electrical pulses may produce unexpectedly beneficial results in the context of tissue ablation. With biphasic electroporation pulses, the direction of the pulses completing one cycle alternates in a few microseconds. As a result, the cells to which the biphasic electrical pulses are applied may undergo alternation of electrical field bias. Changing the direction of bias reduces prolonged post-ablation depolarization and/or ion charging. As a result, prolonged muscle excitation may be reduced. Further, biphasic electrical pulses may overcome the high impedance characteristics of fatty cells that are often problematic in ablation procedures.

In some examples, the pulse width B may be 5 μs or less, based at least in part on the evaluation of bubble output at high voltages and/or evidence of thermal effects on the tissue surface. As for the presence of bubbles, a pulse width of greater than 15 μs may be more likely to produce significant gas bubble volume and pulse widths of 20 μs or longer may produce thermal effects on the tissue surface. No loss of efficacy has been observed when going from 100 μs to 5 μs pulse width. Further, pulses with a pulse width as short as 5 μs may reduce non-collateral tissue stimulation.

An applied voltage amplitude of between approximately 200V and approximately 300V may be the threshold amplitude at which irreversible damage is caused to cells that are in direct contact with the electrodes 22. In general, irreversible electroporative effects may be obtained if the E-field distribution is oriented such that the highest field strength is applied along (or parallel to) the long axis of the targeted cells. However, maximal irreversible electroporative effects may be achieved if multiple field vectors are applied to the targeted cells because different cells may react differently to a particular E-field orientation. The polarity of adjacent electrodes 22 may be alternated to achieve the widest variety of field directions possible. If more than one vector is used, a larger percentage of cells may be affected and a more complete lesion may be created. Although not shown, additional distal portion 20 configurations may be used to produce a variety of E-field vectors. As a non-limiting example, the distal portion 20 may include a mesh-covered balloon, a balloon with embedded surface electrodes, or a splined basket with multiple electrodes. Additionally or alternatively, additional electrodes may be added to existing devices to deliver some of the pulses to add a new field direction.

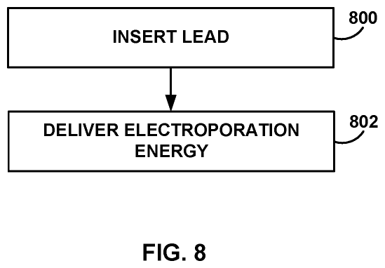

FIG. 8 is a flow diagram of an example technique for using system 10 in accordance with techniques of this disclosure. Lead 14 may be inserted into the body of patient 13 (800). Lead 14 may be navigated to an implantation site (e.g., a location proximate heart 19) within patient 13 such that electrodes 22 are proximate to the implantation site.

When proximate to the implantation site, electrodes 22 may be oriented relative to heart 19 of patient 13. For instance, in examples where electrodes 22 are segmented electrodes (e.g., directional electrodes), electrodes 22 may be oriented toward a posterior sternal surface such that the electrical fields produced by electrodes 22 are simultaneously directed toward target tissue for ablation and away from heart 19.

Lead 14 may deliver electroporation energy to irreversibly electroporate tissue responsible for undesirable sensation (802). For instance, lead 14 may be electrically coupled to electroporation device 35. Electroporation device 35 may include signal generator 64 that provides electrical pulses to electrodes 22 to perform an electroporation procedure. For instance, signal generator 64 may deliver pulsed, high-voltage electric fields appropriate for achieving desired pulsed, high-voltage ablation via IRE and/or pulsed RF ablation.

The pulsed-field energy in accordance with this disclosure may be sufficient to induce cell death for purposes of destroying the ability of the so-ablated tissue to propagate or conduct electrical signals associated with sensation. In this way, electrodes 22 may be configured to deliver electroporation energy to tissue responsible for sensation during pacing, thereby irreversibly electroporating the tissue. The target tissue may include tissue proximate ribcage 15 of patient 13 and anterior to heart 19, such as intercostal nerves, transverse thoracic muscle tissue, etc.

Responsive to completion of IRE, lead 14 may be electrically disconnected from electroporation device 35 and electrically connected to IMD 12.

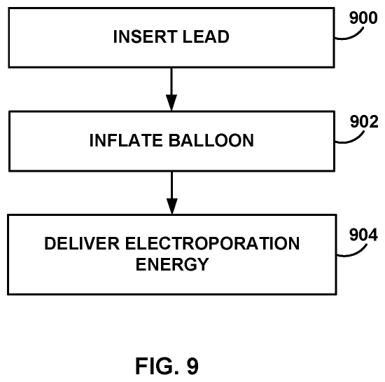

FIG. 9 is a flow diagram of an example technique for using system 10 in accordance with techniques of this disclosure. Lead 14 may be inserted into the body of patient 13 and navigated to an implantation site (900). When proximate to the implantation site, electrodes 22 may be oriented relative to heart 19 of patient 13. Balloon 26 may be inflated (902). When in the inflated configuration, balloon 26 may decrease the distance between electrodes 22 and ribcage 15 and increase the distance between electrodes 22 and heart 19. Consequently, when in an inflated configuration, balloon 26 may position electrodes 22 toward ribcage 15 and away from heart 19, advantageously reducing the likelihood of undesirably electroporating heart 19. Lead 14 may then deliver electroporation energy to electroporate tissue responsible for undesirable sensation (904).

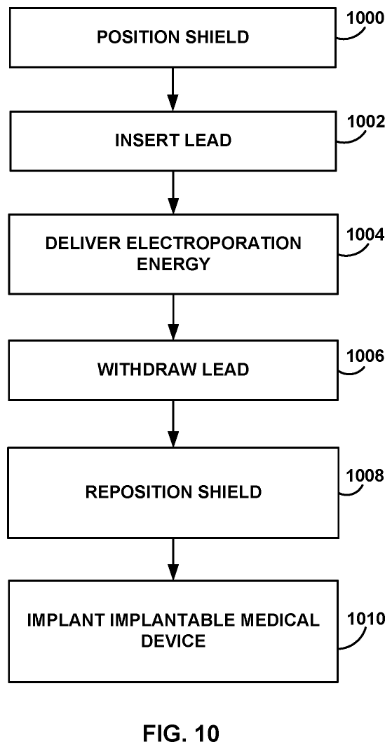

FIG. 10 is a flow diagram of an example technique for using system 10 in accordance with techniques of this disclosure. Shield 42 may be positioned on lead 14 such that when electrodes 22 are navigated to an implantation site and oriented relative to heart 19, shield 42 is disposed between electrodes 22 and heart 19 (1000). Lead 14 may be inserted into the body of patient 13 and navigated to the implantation site (1002). When proximate to the implantation site, electrodes 22 may be oriented relative to heart 19 of patient 13. Lead 14 may deliver electroporation energy to electroporate tissue responsible for undesirable sensation (1004). Lead 14 may be withdrawn from the patient's body (1006). Shield 42 may be repositioned on lead 14 such that when electrodes 22 are navigated to an implantation site and oriented relative to heart 19, electrodes 22 are disposed between shield 42 and heart 19 (1008). IMD 12 may then be implanted with lead 14 secured to the implantation site (1010).

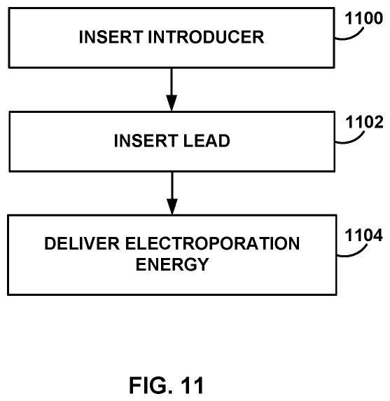

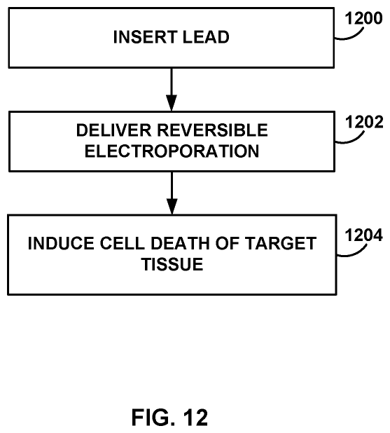

FIG. 11 is a flow diagram of an example technique for using system 10 in accordance with techniques of this disclosure. Introducer 52 may be inserted into the patient's body (1100). Lead 14 may be inserted into the body of patient 13 via introducer 52 and navigated to the implantation site (1102). When proximate to the implantation site, electrodes 22 may be oriented relative to heart 19 of patient 13. Electrodes 22 of lead 14 may deliver electroporation energy to electroporate tissue responsible for undesirable sensation (1104). In some examples, introducer 52 may facilitate deliver electroporation energy to electroporate tissue proximate ribcage 15 and anterior to heart 19. For instance, introducer 52 may deliver electroporation energy through introducer electrodes 56 to electroporate the tissue proximate ribcage 15 and anterior to heart 19. Introducer electrodes 56 may be oriented toward a posterior sternal surface. In some examples, introducer electrodes 56 may be electrically connected to a signal generator, and introducer electrodes 56 may deliver electroporation energy to the target tissue. In other examples, electrodes 22 may be electrically connected to one or more of introducer electrodes 56 via a conductor that conducts the electroporation energy from electrodes 22 to introducer electrodes 56.

In some examples, introducer 52 may define at least one slot 58 that exposes electrodes 22 when electrodes 22 are proximate to the implantation site and oriented relative to heart 19. Additionally or alternatively, introducer 52 may define a porous structure that facilitates delivery of electroporation energy from electrodes 22.

FIG. 12 is a flow diagram of an example technique for using system 10 in accordance with techniques of this disclosure. Lead 14 may be inserted into the body of patient 13 (1200). Lead 14 may be navigated to an implantation site (e.g., a location proximate heart 19) within patient 13 such that electrodes 22 are proximate to the implantation site. When proximate to the implantation site, electrodes 22 may be oriented relative to heart 19 of patient 13. For instance, in examples where electrodes 22 are segmented electrodes (e.g., directional electrodes), electrodes 22 may be oriented toward a posterior sternal surface such that the electrical fields produced by electrodes 22 are simultaneously directed toward target tissue for ablation and away from heart 19.

Lead 14 may deliver electroporation energy to reversibly electroporate tissue responsible for undesirable sensation (1202). Electrodes 22 may be configured to deliver electroporation energy to tissue responsible for sensation during pacing, thereby reversibly electroporating the tissue. The target tissue may include tissue proximate ribcage 15 of patient 13 and anterior to heart 19, such as intercostal nerves, transverse thoracic muscle tissue, etc. The clinician may confirm that the desired effect (e.g., no stimulation) has been temporarily achieved due to electroporation of the target tissue. Responsive to confirmation of the target tissue, lead 14 may deliver IRE or a therapy agent to cause cell death in the reversibly electroporated cells. In some examples, the therapy agent may be preferentially placed on lead 14 to target the side of lead 14 closer to ribcage 15 of patient 13. Additionally or alternatively, a local or systemic injection of a therapy agent may cause cell death following the reversible electroporation energy, allowing for the same ablative effect.

The following examples are illustrative of the techniques described herein.

Example 1: A medical system includes a medical device includes an elongated structure configured to be navigated from an access point of a patient to an implantation site within the patient; and at least one electrode carried on a distal portion of the elongated structure, wherein the at least one electrode is configured, when proximate to the implantation site, to be oriented relative to a heart of the patient, and wherein the at least one electrode is configured to deliver electroporation energy to electroporate tissue proximate a ribcage of the patient and anterior to the heart.

Example 2: The medical system of example 1, wherein the at least one electrode includes one or more segmented electrodes configured to be oriented toward a posterior sternal surface.

Example 3: The medical system of example 1 or 2, further including a balloon configured to, when in an inflated configuration, position the at least one electrode toward the ribcage and away from the heart.

Example 4: The medical system of any of examples 1 to 3, further including a shield configured to impede delivery of electroporation energy from the at least one electrode toward the heart.

Example 5: The medical system of any of examples 1 to 4, wherein the tissue includes at least one of muscle tissue or nerve tissue.

Example 6: The medical system of any of examples 1 to 5, wherein the elongated structure is a catheter, an introducer, or an implantable medical lead.

Example 7: The medical system of any of examples 1 to 6, wherein the at least one electrode is configured to deliver electroporation energy to irreversibly electroporate the tissue or to reversibly electroporate the tissue.

Example 8: The medical system of any of examples 1 to 7, wherein elongated structure is further configured to deliver a therapy agent to the tissue, wherein the therapy agent includes at least one of an anti-inflammatory agent, an analgesic agent, a neurotoxin, or an antimicrobial agent.

Example 9: The medical system of example 8, wherein the therapy agent includes Bleomyocin.

Example 10: The medical system of example 8 or 9, wherein the therapy agent is preferentially placed on the elongated structure to target the side of the elongated structure closer to the ribcage of the patient.

Example 11: The medical system of any of examples 1 to 10, further including an introducer defining a lumen, wherein the elongated structure is configured to be navigated from the access point to the implantation site within the patient using the introducer, wherein the lumen is sized to allow insertion of the implantable medical lead into the introducer.

Example 12: The medical system of example 11 wherein the at least one electrode carried on the distal portion of the implantable medical lead includes at least one lead electrode, and wherein the introducer includes at least one introducer electrode configured to deliver electroporation energy to electroporate the tissue proximate the ribcage and anterior to the heart.

Example 13: The medical system of example 12, wherein the introducer further includes a conductor configured to conduct the electroporation energy from the at least one lead electrode to the at least one introducer electrode.

Example 14: The medical system of any of examples 11 to 13, wherein the introducer defines at least one slot that exposes the at least one electrode of the implantable medical lead when the at least one electrode is proximate to the implantation site and oriented relative to the heart of the patient.

Example 15: The medical system of any of examples 11 to 14, wherein the introducer defines a porous structure that facilitates delivery of electroporation energy from the at least one electrode.

Example 16: The medical system of any of examples 1 to 15, further includes configure the electroporation energy to electroporate the tissue; and deliver the electroporation energy to the at least one electrode of the medical device.

Example 17: A medical device includes an elongated structure configured to be navigated from an access point of a patient to an implantation site within the patient; and at least one electrode carried on a distal portion of the elongated structure, wherein the at least one electrode is configured, when proximate to the implantation site, to be oriented relative to a heart of the patient, and wherein the at least one electrode is configured to deliver electroporation energy to irreversibly electroporate tissue proximate a ribcage of the patient and anterior to the heart.

Example 18: The medical device of example 17, wherein the at least one electrode includes one or more segmented electrodes configured to be oriented toward a posterior sternal surface.

Example 19: The medical device of example 17 or 18, further including a balloon configured to, when in an inflated configuration, position the at least one electrode toward the ribcage and away from the heart.

Example 20: The medical device of any of examples 17 to 19, further including a shield configured to impede delivery of electroporation energy from the at least one electrode toward the heart.

Example 21: The medical device of any of examples 17 to 20, wherein the tissue includes at least one of muscle tissue or nerve tissue.

Example 22: The medical device of any of examples 17 to 21, wherein the elongated structure is a catheter, an introducer, or an implantable medical lead.

Example 23: A method includes delivering, by at least one electrode carried on a distal portion of an elongated structure navigated from an access point of a patient to an implantation site within the patient, electroporation energy to irreversibly electroporate tissue proximate a ribcage of the patient and anterior to the heart, wherein the at least one electrode is oriented relative to the heart of the patient.

Example 24: The method of example 23, further including inflating a balloon to position the at least one electrode toward the ribcage and away from the heart.

Example 25: The method of example 23 or 24, further including positioning a shield relative to the at least one electrode such that when, the at least one electrode delivers electroporation energy, the shield is disposed between the at least one electrode and the heart.

Example 26: The method of any of examples 23 to 25, further including inserting an introducer into the access point of the patient, wherein navigating the elongated structure from the access point to the implantation site includes inserting the elongated structure into the introducer.

Example 27: The method of any of examples 23 to 26, wherein the introducer includes at least one introducer electrode configured to deliver electroporation energy to irreversibly electroporate the tissue proximate the ribcage and anterior to the heart.

Example 28: A medical system includes a medical device includes an elongated structure configured to be navigated from an access point of a patient to an implantation site within the patient; and at least one electrode carried on a distal portion of the elongated structure, wherein the at least one electrode is configured, when proximate to the implantation site, to be oriented relative to a heart of the patient, and wherein the at least one electrode is configured to deliver electroporation energy to irreversibly electroporate tissue proximate a ribcage of the patient and anterior to the heart; and an electroporation device configured to: configure the electroporation energy to irreversibly electroporate the tissue; and deliver the electroporation energy to the at least one electrode of the medical device.

Example 29: The medical system of example 28, wherein the at least one electrode includes one or more segmented electrodes configured to be oriented toward a posterior sternal surface.

Example 30: The medical system of example 28 or 29, further including a balloon configured to, when in an inflated configuration, position the at least one electrode toward the ribcage and away from the heart.

Example 31: The medical system of any of examples 28 to 30, further including a shield configured to impede delivery of electroporation energy from the at least one electrode toward the heart.

Example 32: The medical system of any of examples 28 to 31, wherein the tissue includes at least one of muscle tissue or nerve tissue.

Example 33: The medical system of any of examples 28 to 32, wherein the elongated structure is a catheter, an introducer, or an implantable medical lead.

Example 34: The medical system of any of examples 28 to 33, further including an external electrode configured to be placed proximate a sternum of the patient, wherein the at least one electrode includes one or more circumferential electrodes.

Various aspects of the disclosure have been described. These and other aspects are within the scope of the following claims.

1. A medical system comprising:
a medical device comprising:
an elongated structure configured to be navigated from an access point of a patient to an implantation site within the patient; and
at least one electrode carried on a distal portion of the elongated structure, wherein the at least one electrode is configured, when proximate to the implantation site, to be oriented relative to a heart of the patient, and wherein the at least one electrode is configured to deliver electroporation energy to electroporate tissue proximate a ribcage of the patient and anterior to the heart.

2. The medical system of claim 1, wherein the at least one electrode comprises one or more segmented electrodes configured to be oriented toward a posterior sternal surface.

3. The medical system of claim 1, further comprising a balloon configured to, when in an inflated configuration, position the at least one electrode toward the ribcage and away from the heart.

4. The medical system of claim 1, further comprising a shield configured to impede delivery of electroporation energy from the at least one electrode toward the heart.

5. The medical system of claim 1, wherein the tissue comprises at least one of muscle tissue or nerve tissue.

6. The medical system of claim 1, wherein the elongated structure is a catheter, an introducer, or an implantable medical lead.

7. The medical system of claim 1, wherein the at least one electrode is configured to deliver electroporation energy to irreversibly electroporate the tissue or to reversibly electroporate the tissue.

8. The medical system of claim 1, wherein elongated structure is further configured to deliver a therapy agent to the tissue, wherein the therapy agent comprises at least one of an anti-inflammatory agent, an analgesic agent, a neurotoxin, or an antimicrobial agent.

9. The medical system of claim 8, wherein the therapy agent comprises Bleomyocin.

10. The medical system of claim 8, wherein the therapy agent is preferentially placed on the elongated structure to target the side of the elongated structure closer to the ribcage of the patient.

11. The medical system of claim 1, further comprising an introducer defining a lumen, wherein the elongated structure is configured to be navigated from the access point to the implantation site within the patient using the introducer, wherein the lumen is sized to allow insertion of the implantable medical lead into the introducer.

12. The medical system of claim 11 wherein the at least one electrode carried on the distal portion of the implantable medical lead comprises at least one lead electrode, and wherein the introducer comprises at least one introducer electrode configured to deliver electroporation energy to electroporate the tissue proximate the ribcage and anterior to the heart.

13. The medical system of claim 12, wherein the introducer further comprises a conductor configured to conduct the electroporation energy from the at least one lead electrode to the at least one introducer electrode.

14. The medical system of claim 11, wherein the introducer defines at least one slot that exposes the at least one electrode of the implantable medical lead when the at least one electrode is proximate to the implantation site and oriented relative to the heart of the patient.

15. The medical system of claim 11, wherein the introducer defines a porous structure that facilitates delivery of electroporation energy from the at least one electrode.

16. The medical system of claim 1, further comprising an electroporation device configured to:
configure the electroporation energy to electroporate the tissue; and
deliver the electroporation energy to the at least one electrode of the medical device.

17. A method comprising:
delivering, by at least one electrode carried on a distal portion of an elongated structure navigated from an access point of a patient to an implantation site within the patient, electroporation energy to irreversibly electroporate tissue proximate a ribcage of the patient and anterior to the heart,
wherein the at least one electrode is oriented relative to the heart of the patient.

18. The method of claim 17, further comprising inflating a balloon to position the at least one electrode toward the ribcage and away from the heart.

19. The method of claim 17, further comprising positioning a shield relative to the at least one electrode such that when, the at least one electrode delivers electroporation energy, the shield is disposed between the at least one electrode and the heart.

20. The method of claim 17, further comprising inserting an introducer into the access point of the patient, wherein navigating the elongated structure from the access point to the implantation site comprises inserting the elongated structure into the introducer.
